(19) United States
(12) Patent Application Publication
(10) Pub. No.: US 2001/0049454 A1
(43) Pub. Date: Dec. 6, 2001

(54) Prepolymer composition for producing insulating foams with increased foam yield

(75) Inventors: Rainer Nutzel, Weissbad (CH); Rene Schumacher, Goldach (CH)

Correspondence Address: REED SMITH HAZEL THOMAS LLP, Suite 1400, 3110 Fairview Park Drive, Falls Church, VA, 22042, US

(73) Assignee: Rathor AG.

(21) Appl. No.: 09/899,271

(22) Filed: Jul. 6, 2001

Related U.S. Application Data
(63) Continuation-in-part of application No. 09/468,126, filed on Dec. 21, 1999.

(30) Foreign Application Priority Data
Aug. 23, 1999 (DE) 199 39 926.3

(51) Int. Cl. C07C 261/00; C08J 9/00

(52) U.S. Cl. 560/25; 560/26; 560/330

(57) ABSTRACT

The invention relates to a prepolymer composition, in particular for producing insulating foams, based on a polyisocyanate with a functionality of at least 2 and a polyol with at least two OH groups in the molecule, the prepolymer having an isocy-anate content of 6 to 20 wt % and the composition containing usual additives and a propellant gas component which is liquid under pressure, and the prepolymer com-position containing at least 18 wt % of propellant gas based on the weight of the composition, said propellant gas component being present in the form of a propellant phase emulsified in the prepolymer composition alongside the moiety dissolved in the prepolymer composition.

This invention relates to a prepolymer composition, in particular for producing insulating foams, based on a polyisocyanate with a functionality of at least 2 and a polyol with at least two OH groups in the molecule, the prepolymer having an isocy-anate content of 6 to 18 wt % and the composition containing usual additives and a propellant gas component which is liquid under pressure.

The inventive prepolymer composition serves to produce polyurethane insulating foams used mainly for foaming cavities. The main field of application is civil engineering. However, it is also used for foaming technical products in which cavities must be filled to avoid condensation nests.

As far as one-component polyurethane foams are concerned, they are discharged by discharging the prepolymer composition from pressurized containers, for example aerosol cans, using propellants in situ with a density of 10 to 50 g/l and processed. One-component foams are moisture curing, i.e. they can cure solely with the moisture in the air, whereby released CO₂ supports the foaming process.

Two-component polyurethane foams require for the prepolymer composition to be cured a second component, usually one containing hydroxyl groups, normally a polyol, which must be added directly before foam formation. Curing can be accelerated by catalysts. Densities in two-component foams are typically 10 to 100 g/l. The second component can contain water as a crosslinker. The CO₂ resulting from the reaction with water supports foam formation.

Transitional forms between one-component and two-component foams as described above are usual. In this case one adds to the prepolymer before discharge a quantity of a hydroxyl component insufficient for reacting the free isocyanate groups contained therein. These transitional forms have become known as “1.5-component foams.” They also include compositions having more than one separate reactive component.

The present invention relates in particular to the area of one-component and 1.5-component pressure-can foams which foam after being released from the aerosol can with the aid of propellant gas and CO₂ formed by chemical reaction. However, the invention can also be used in conjunction with two-component foams.

The essential criterion for assessing the economy of prepolymer compositions for producing insulating foams is their yield. In the normal case one can assume that a good prepolymer composition from a one-liter one-component pressure can which is 75% full will yield about 50 liters of foam. A further quality feature is that the foam has low shrinkage and good insulating quality.

Foam formation in one-component pressure-can foams is due to a number of interacting processes, cell expansion first being effected through evaporation of dissolved propellant, this being intensified by thermal effects due to heating. Reaction of the prepolymer with moisture in the air causes CO₂ to be released, which exerts an additional blowing effect. Reaction with moisture simultaneously causes the prepolymer to be cured and thus stabilizes the foam skeleton under the expansion pressure.

Foam yield is naturally subject to limits by this concept. Limits are set by the ability of the prepolymer to entrain and release propellant and the quantity of free isocyanate groups which react exhaustively with air moisture so as to form CO₂. It is to be taken into account that the normally nonpolar propellant gases are not soluble in the prepolymer composition without limit and that only a limited quantity of free isocyanate groups is available for reacting with water because of the preadjustment of the foam with polyols to desired properties.

It would fimdamentally be desirable to increase foam yield from a given quantity of prepolymer, firstly for reasons of cost and secondly for technical reasons. The quality of a thermal insulating foam depends on the quantity of enclosed air, which brings about the actual insulation A greater volume of foam is thus synonymous with lower foam density and higher insulating power.

It has now surprisingly been found that the foam yield of usual prepolymer compositions of the abovementioned kind can be increased by up to 30 vol % if the prepolymer composition contains at least 18 wt % of propellant gas based on the weight of the composition and the propellant gas component forms a separate propellant phase emulsifiable in the composition under pressure alongside the moiety dissolved in the prepolymer composition.

The invention can be realized with usual prepolymer compositions of the abovementioned kind which contain at least 18 wt % of propellant gas based on the weight of the composition, the propellant gas component being only partly dissolved in the prepolymer composition and additionally forming a propellant phase emulsifiable in the composition.

The separate propellant phase in the inventive prepolymer composition expediently constitutes at least 15 wt % of the propellant gas component and preferably at least 20 wt %.

The inventive prepolymer composition forms a two-phase system in a conventional pressure or aerosol can, whereby the second phase, i.e. the excess propellant over the propellant gas dissolved in the composition, is emulsified under the system pressure in the first phase. As has turned out, the two-phase system normally remains stable or at least semistable under pressure. Semistable means that the emulsion can be produced from the two components anytime by shaking or other suitable measures and then persists for some time before the components separate again.

Emulsifiers as are normally already contained in pressure-can compositions as stabilizers can have a supporting effect but are not absolutely necessary according to the invention, in particular if the prepolymer composition itself and the propellant mixture used have roughly the same density. Of the emulsifiers/stabilizers normally used, those with hydrophobic properties are preferred.

According to the invention the propellant component constitutes at least 18 wt %, preferably at least 20 wt % and in particular at least 22 wt %, of the prepolymer composition.

Experimental results have shown that the increased quantity of propellant gas component entails a substantial increase in foam yield. It was found that merely increasing the propellant moiety in a conventional prepolymer composition increased foam yield from a standard 50 liters to altogether 65 liters, i.e. by 30%. With other commercial compositions it was at least 20%. In experiments in pressurized inspection glasses it was found that a second propellant gas phase forms in the aerosol can, being emulsified in the prepolymer phase but possibly separating after standing for a while. Thorough shaking will restore the emulsion even after a relatively long storage period. The formation of a separate solvent phase and the emulsifiability of this phase are preconditions for increased foam yield It was ascertained that the hydro-carbons/ethers are enriched in the solvent phase.

The mode of action of the second propellant phase can be explained by a preexpansion of the foam occurring after discharge of the foam with the propellant gas droplets emulsified therein, before actual normal foam formation begins via cell expansion (through evaporation of the dissolved propellant gas moiety), thermal effects (through heating of the propellant gas and the resulting expansion) and curing of the prepolymer (through crosslinking with water and release of CO₂). Surprisingly, it was further found that this preexpansion effected according to the invention involves no disadvantages with respect to the dimensional stability of the produced foam. One would normally expect the preexpansion, which takes place in a stage when the foam is not yet cured, not to lead to a lasting volume expansion.

The propellant gases preferably used according to the invention are a combination of a fluorocarbon, on the one hand, and a hydrocarbon and/or ether, on the other hand, it being evident that the propellant gases at least predominantly have a boiling point below room temperature and are liquefiable under pressure and are at most partly low-boiling with a boiling point below 50C. The propellant gas mixture should have roughly the same density as the prepolymer composition, i.e. not deviate downward or upward therefrom by more than about 10%. Normally, the separate solvent phase has a lower density than the prepolymer composition.

Suitable fluorocarbons are for example R 134a, R 152a and R 125, whereby R 134a is especially preferred. Suitable hydrocarbons are in particular propane, n-butane, isobutane, n-pentane and isopentane. Propane and the butanes are liquefiable under pressure. Suitable ethers are in particular dimethyl ether and diethyl ether, the former being liquefiable under pressure.

A particularly preferred fluorocarbon is CF₃CH₂F(R 134a) which has limited solubility in polyurethane prepolymers.

Besides the stated propellant gases further propellants can be present, e.g CO₂ as a permanent gas.

An especially preferred propellant gas composition is a mixture of R 134a with dimethyl ether and propane, butane and/or pentane. The moiety of propellant gases with a boiling point above room temperature should constitute no more than 25 wt % of the propellant gas components.

With respect to the propellant gases which are liquid at room temperature it is to be noted that crosslinking of the prepolymer takes place under heat buildup so that the temperature increase and resulting delay in evaporation of the higher-boiling propellant gas component cause stronger postexpansion. The expansion process thus lasts for the total time period from discharge of the prepolymer to final curing. This also counteracts subsequent shrinkage of the foam.

The ratio of fluorocarbon to hydrocarbon/ether in the propellant gas component is expediently 40:60 to 80:20 by weight. An especially preferred weight ratio of fluorocarbon to hydrocarbon/ether is 60:40 to 75:25. The fluorocarbon constitutes at least 13 wt % of the prepolymer composition.

As mentioned, conventional prepolymer compositions can be used according to the invention. These conventional prepolymer compositions have a minimum content of reactive NCO groups which is in the range of 6 to 18 wt %. The prepolymer itself is a polymer of suitable viscosity with terminal NCO groups based on a suitable polyisocyanate. Suitable polyisocyanates are for example isophorone diisocyanate, also known as IPDI, toluene diisocyanate, also known as TDI, 1,5-naphthalene diisocyanate, also known as NDL trimethylmethane triisocyanate, 1,6-hexane diisocyanate and 4,4-diphenylmethane diisocyanate, usually known as MDI. All these staring materials can be used in raw form, i.e. as a mixture, as well as in the form of the pure isomers or as mixtures of the isomers or in the form of their reactive derivatives. As reactive derivatives one can use mainly dimeric and trimeric forms of the isocyanates as well as their biurets and allophanates. A functionality of at least 2 is important.

For producing the prepolymers one reacts such polyisocyanates with polyethers, polyesters or polyvalent alcohols containing hydroxyl groups, making sure that the resulting prepolymer acquires a viscosity suitable for the purpose. The reaction takes place in the presence of a catalyst which brings about or accelerates the desired reaction. The catalyst is normally based on amines, or based on carboxylates.

Usually, such prepolymer compositions for producing insulating foams contain a number of additives intended to influence the properties of the prepolymer composition as well as the foam produced therefrom. Such additives are not only catalysts for the crosslinking reaction but also for example stabilizers, plasticizers and flameproofing agents, whereby in particular the plasticizers and flameproofing agents can at the same time have crosslinking properties and the stabilizers normally have an emulsifying effect. Suitable stabilizers are based on silicone, as sold by Goldschmidt AG in Essen under the designation “Tegostab”®. Hydrophobic silicone stabilizers are preferred.

The polyol component used for the inventive compositions can be the usual components containing hydroxyl groups, for example polyester polyols, polyether polyols and natural or modified vegetable oils. It is especially preferred to use polyester polyols and in particular natural or modified vegetable oils. A natural vegetable oil to be used is for example castor oil, having hydroxyl groups reactive with NCO groups. Modified vegetable oils to be used, for example modified castor oil, are in particular transesterification products, having the necessary number of OH groups, in particular their transesterification products with glycerol and other low-molecular polyols.

Generally, suitable polyols have an OH number in the range of 1 to 300 and at least two OH groups in the molecule.

Catalysts to be used for producing the prepolymer compositions according to the invention are capable of catalyzing the reaction of isocyanate groups with hydroxyl groups. Especially preferred catalysts are based on tertiary amines, for example N-methyl-2-azanorbornane, triethylenediamine, dimorpholinodiethyl ether (DMDEE), trimethylaminoethyl piperazine, pentamethyldiethylene triamine, tetra-methyliminobispropylamine and bis(dimethylaminopropyl)-N-isopropanolamine.

The inventive prepolymers generally have a content of free NCO groups of 6 to 18 wt %, preferably 10 to 15 wt %, based on the prepolymer itself It is expedient to adjust the viscosity of the prepolymer, including all additives but without propellant gas, to an initial service viscosity at 20° C. of 5,000 to 20,000 mPas and preferably 8,000 to 15,000 mPas.

The inventive prepolymer compositions are produced in the usual way and filled into usual in-situ foam pressure cans. They can be discharged as one-, 1.5- or two-component systems. 1.5- and two-component systems require the presence in the pressure can of a crosslinker tube capable of discharging, when triggered, a crosslinker into the mixture in a quantity sufficient to react with the diisocyanate groups present in the prepolymer partly (1.5-component) or completely (two-component).

The inventive prepolymer compositions are produced in the way known in the art, whereby the prepolymer can be produced both in the pressure can and outside. One then adds to the prepolymer in the pressure can the required additives, e.g. stabilizers, cell openers and flameproofing agents, optionally also surfactants and further polyol for finely adjusting NCO content. The pressurized container (aerosol can) is then sealed and the propellant impressed.

The invention will be explained by the following examples.

### EXAMPLES 1 to 3

Prepolymer compositions were produced with the components shown in the following table in the stated quantities. The polyisocyanate used was raw MDI (Desmodur® from Bayer AG in Leverkusen). The polyol component used was a polyether polyol (Voranol® from Dow Chemical) with OH number 160 and a polyester polyol (Stepanpol® from Stepan, Wesseling, DE) with OH number 170. All mixtures contained stabile based on silicone and DMDEE as a catalyst. The flameproofing agent used was bromophthalic acid ester (Isoexter® from Coim, Settimo Milanese, IT).

All mixtures contained a propellant gas mixture of fluorocarbon, hydrocarbons and dimnethyl ether.

The relevant data of the composition as well as foam yield are rendered in the following table.

| | EX. 1 | EX. 2 | EX. 3 |
|---|---|---|---|
| Voranol CP 1055 | 120 | 412 | 560 |
| Stepanpol PS-1752 | 390 | | |
| Tris(monochloropropyl)phosphate | 430 | 440 | 395 |
| Isoexter 3510 | | 108 | |
| Tegostab P 8870 | 50 | 30 | 30 |
| DMDEE | 10 | 10 | 15 |
| Total | 1000 | 1000 | 1000 |
| Polyol mixture* | 315 | 286 | 281 |
| Desmodur 44 V 20 L | | | 332 |
| Desmodur 44 V 10 L | 339 | 350 | |
| R 134a | 112 | 134 | 149 |
| Isobutane | 30 | | 39 |
| Dimethyl ether | 32 | 20 | 19 |
| Isopentane | | 43 | |
| Total weight in g | 828 | 833 | 819 |
| Total volume in ml | 750 | 750 | 750 |
| Propellant gas moiety incl. pentane in vol % | 26 | 28 | 29 |
| Propellant gas moiety incl. pentane in wt % | 21 | 23 | 25 |
| Excess NCO in % | 12.99 | 13.90 | 14.04 |
| Filling degree of can in vol % | 75% | 75% | 75% |
| Foam yield | 591 | 611 | 651 |

*as above

1. A prepolymer composition, in particular for producing insulating foams, based on a polyisocyanate with a functionality of at least 2 and a polyol with at least two OH groups in the molecule, the prepolymer having an isocyanate content of 6 to 18 wt % based on the prepolymer, and the composition containing usual additives and a propellant gas component which is liquid under pressure, characterized in that the prepolymer composition contains at least 18 wt % of propellant gas based on the weight of the composition, the propellant gas component being dimensioned such that a propellant phase emulsified in the prepolymer composition is present alongside propellant gas dissolved in the prepolymer composition.

2. A prepolymer composition according to claim 1, characterized in that it contains at least 20 wt % of propellant gas based on the total prepolymer composition.

3. A prepolymer composition according to claim 2, characterized in that it contains at least 22 wt % of propellant gas based on the prepolymer composition.

4. A prepolymer composition according to any of the above claims, characterized in that the propellant gas component contains a fluorocarbon together with a hydrocarbon and/or the low-boiling ether.

5. A prepolymer composition according to claim 4, characterized in that the propellant phase has roughly the same density as the prepolymer composition.

6. A prepolymer composition according to claim 4 or 5, characterized in that the fluorocarbon is CF₃CH₂F(R 134a).

7. A prepolymer composition according to claim 6, characterized in that the propellant gas component contains propane, isobutane, n-butane and/or dimethyl ether.

8. A prepolymer composition according to any of claims 4 to 7, characterized in that the propellant gas component additionally contains isopentane and/or n-pentane.

9. A prepolymer composition according to any of claims 4 to 8, characterized in that the weight ratio of fluorocarbon to hydrocarbon and/or ether is in the range of 40:60 to 80:20.

10. A prepolymer composition according to claim 9, characterized in that the weight ratio is in the range of 60:40 to 75:25.

11. A prepolymer composition according to any of the above claims, characterized in that at least 15 wt % of the propellant gas component is present as a separate phase.

12. A prepolymer composition according to any of claims 4 to 11, characterized in that it contains the fluorocarbon in a quantity of at least 13 wt % based on the total composition.

13. A prepolymer composition according to any of the above claims, characterized in that it contains alongside the prepolymer plasticizers, flameproofing agents, stabilizers, cell openers, cell regulators, catalysts, carbon solvents and/or emulsifiers.

14. A prepolymer composition according to any of the above claims, characterized in that it contains a hydrophobic silicone stabilizer in a quantity of 0.5 to 3.0 wt % based on the total composition.

15. A prepolymer composition according to any of the above claims, contained in a pressure can.
